SEQUENCE LISTING (14 sequences)

SEQ ID NO: 1 - protein, 9 aa, Artificial Sequence
  chemically-synthesized histidine-lysine peptides
KHKHKHKHK

SEQ ID NO: 2 - protein, 10 aa, Artificial Sequence
  chemically-synthesized histidine-lysine peptides
HKHKHKHKHK

SEQ ID NO: 3 - protein, 10 aa, Artificial Sequence
  chemically-synthesized histidine-lysine peptides
KHKHKHKHKH

SEQ ID NO: 4 - protein, 11 aa, Artificial Sequence
  chemically-synthesized histidine-lysine peptides
HKHKHKHKHKH

SEQ ID NO: 5 - protein, 20 aa, Artificial Sequence
  chemically-synthesized histidine-lysine peptides
KHKHHKHHKHHKHHKHHKHK

SEQ ID NO: 6 - protein, 17 aa, Artificial Sequence
  chemically-synthesized histidine-lysine peptides
KHHHKHHHKHHHKHHHK

SEQ ID NO: 7 - protein, 18 aa, Artificial Sequence
  chemically-synthesized histidine-lysine peptides
KHHHKHHHKHHHHKHHHK

SEQ ID NO: 8 - protein, 18 aa, Artificial Sequence
  chemically-synthesized histidine-lysine peptides
KHHHKHHHKHHHHKHHHK

SEQ ID NO: 9 - protein, 19 aa, Artificial Sequence
  chemically-synthesized histidine-lysine peptides
HKHHHKHHHKHHHHKHHHK

SEQ ID NO: 10 - DNA, 20 bp, Artificial Sequence
  chemically-synthesized histidine-lysine peptides
hhkhhhkhhhkhhhhkhhhk

SEQ ID NO: 11 - protein, 21 aa, Artificial Sequence
  chemically-synthesized histidine-lysine peptides
KHHHHKHHHHKHHHHKHHHHK

SEQ ID NO: 12 - protein, 18 aa, Artificial Sequence
  chemically-synthesized histidine-lysine peptides
KHHHKHHHKHHHKHHHHK

SEQ ID NO: 13 - protein, 18 aa, Artificial Sequence
  chemically-synthesized histidine-lysine peptides
KHHHKHHHHKHHHKHHHK

SEQ ID NO: 14 - protein, 19 aa, Artificial Sequence
  chemically-synthesized histidine-lysine peptides
KHHHKHHHHKHHHKHHHHK

(19) United States
(12) Patent Application Publication
(10) Pub. No.: US 2023/0313181 A1
(43) Pub. Date: Oct. 5, 2023

(54) HISTIDINE-LYSINE POLYMERS AND METHODS FOR DELIVERING MRNA USING THE SAME

(71) Applicants: Archibald James MIXSON, Rockville, MD (US); Jiaxi HE, Baltimore, MD (US)

(72) Inventors: Archibald James MIXSON, Rockville, MD (US); Jiaxi HE, Baltimore, MD (US)

(73) Assignee: UNIVERSITY OF MARYLAND, BALTIMORE, Baltimore, MD (US)

(21) Appl. No.: 17/787,780

(22) PCT Filed: Dec. 23, 2020
(86) PCT No.: PCT/US20/66878

§ 371 Date: Jun. 21, 2022

Related U.S. Application Data
(60) Provisional application No. 62/952,727, filed on Dec. 23, 2019.

(51) Int. Cl. C12N 15/113; A61K 47/64; A61K 47/56; A61K 47/54; A61K 9/107

(52) U.S. Cl. CPC C12N 15/113; A61K 47/6455; A61K 47/56; A61K 47/543; A61K 9/1075

(57) ABSTRACT

Compositions and methods for improved delivery of mRNA into eukaryotic cells using histidine-lysine (HK) peptide polymers are disclosed. The branched polymers comprising four short peptide branches linked to a three-lysine amino acid core. The peptide branches consist of histidine and lysine amino acids, in different configurations, and they can vary in their location on the lysine core.

### SEQUENCE LISTING

A sequence listing in electronic (ASCII text file) format is filed with this application and incorporated herein by reference. The name of the ASCII text file is “2020_2755A_ST25.txt”; the file was created on Dec. 21, 2020; the size of the file is 5 KB.

### TECHNICAL FIELD

The invention relates to the fields of medicine and molecular biology, and it is directed to compositions and methods for improved delivery of mRNA into eukaryotic cells. In particular, the invention relates to histidine-lysine (HK) peptide carriers which exhibit enhanced cellular mRNA transfection efficiency.

### BACKGROUND OF INVENTION

Direct delivery of mRNA to target cells is an improvement over the use of plasmids-based delivery systems because direct delivery allows translation to a protein in the cytosol of the cell without requiring entry of the polynucleotide into the nucleus in order to become functional. As a result of cytosolic translation, successful expression of proteins can be achieved in non-dividing cells [1]. Although degradability of mRNA may in some ways be advantageous, e.g. to reduce toxicity [2, 3], the susceptibility of mRNA to enzymatic degradation with reduced translation accounts for significant problems. Consequently, the development of carriers that can protect mRNA from degradation, facilitate cellular uptake, and enhance buffering capacity to improve endosomal escape has become a high priority. Among potential candidates for next generation delivery systems are non-viral carriers, including polymers and lipid-based agents including lipopolymers and liposomes, that have shown some utility in mRNA delivery [4-8]. Of these, liposomes are the most studied and they are effective carriers of mRNA [1, 9-13]. For instance, Zohra et al. found that DOTAP liposomes coated with carbonate apatite exhibited high luciferase mRNA transfection efficiency in both mitotic and non-mitotic cells [1, 13].

There have been only a limited number of studies demonstrating the utility of polymers as mRNA carriers [6-8, 14-25]. Qiu and colleagues synthesized an RNA delivery vector, PEG12KL4, in which the synthetic cationic KL4 peptide was attached to a linear 12-mer of PEG. With intratracheal administration, these carriers mediated significantly more effective mRNA transfection in the lungs of mice than naked mRNA [23]. Moreover, based on the studies of Kataoka and co-workers [14], Chan et al. compared several repeating units of aminoethylene groups (2, 3, or 4) conjugated as side chains to a PEGylated polyaspartamide backbone [24]. The carrier with the side branch of four-repeating units, tetraethylenepentamine, had the best luciferase mRNA delivery efficiency in vitro and effectively delivered luciferase mRNA injected intracerebroventricular with no significant immune response. Interestingly, by altering the alkyl length between amines, the group of Dohmen found an oligoalkylamine that significantly enhanced mRNA expression [6]. This oligoalkylamine had a high buffering capacity between pH 6.2 and 6.5, a pH range that has been associated with endosomal lysis and escape of nucleic acids. Several investigators have also utilized either peptide-liposomes or lipopolymers to stabilize the vector to deliver mRNA in vivo. With few exceptions [15, 17], lipid-polymer hybrids or liposome-polymer combinations are required or at least greatly enhance systemic delivery of mRNA [6-8, 21, 22, 26].

The development of new mRNA carrier systems thus continues to be an important unmet need. The preset invention is directed to devising such carriers systems and other related and important goals.

### BRIEF SUMMARY OF INVENTION

The invention relates to branched polymers comprising four short peptide branches linked to a three-lysine amino acid core. The peptide branches consist of histidine and lysine amino acids, in different configurations, and they can vary in their location on the lysine core.

Thus, and in a first embodiment, the invention is directed to histidine-lysine peptide polymers (HK polymers) of Formula I and II, where K is L-lysine and each of R₁, R₂, R₃ and R₄ is independently (i) KHₙKHₙKHₙKHₙK— (SEQ ID NO:1), (ii) HₙKHₙKHₙKHₙKHₙK— (SEQ ID NO:2), (iii) KHₙKHₙKHₙKHₙKHₙ— (SEQ ID NO:3), or (iv) HₙKHₙKHₙKHₙKHₙKHₙ— (SEQ ID NO:4), wherein in (i), (ii), (iii) and (iv) each H is L-histidine or D-histidine, each K is L-lysine or D-lysine, and each n is independently an integer of between 0 and 4.

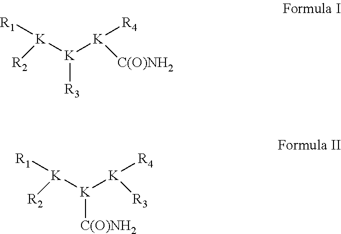

The R₁₋₄ branches may be the same or different in the HK polymers of the invention. Thus, the HK polymers of the invention include polymers where each of R₁, R₂, R₃, and R₄ are the same; where each of R₁, R₂, R₃, and R₄ are different; where R₁ is different and R₂, R₃ and R₄ are the same; where R₂ is different and R₁, R₃ and R₄ are the same; where R₃ is different and R₁, R₂ and R₄ are the same; where R₄ is different and R₁, R₂ and R₃ are the same; where R₁ and R₂ are the same, and R₃ and R₄ are different; where R₁ and R₂ are different, and R₃ and R₄ are the same; where R₁ and R₂ are the same, and R₃ and R₄ are the same; where R₁ and R₃ are the same, and R₂ and R₄ are different; where R₁ and R₃ are different, and R₂ and R₄ are the same; where R₁ and R₃ are the same, and R₂ and R₄ are the same; where R₁ and R₄ are the same, and R₂ and R₃ are different; where R₁ and R₄ are different, and R₂ and R₃ are the same; and where R₁ and R₄ are the same, and R₂ and R₃ are the same. When a R branch is “different”, the amino acid sequence of that branch differs from each of the other R branches in the polymer.

Suitable R branches used in the HK polymers of the invention shown in Formula I and II include, but are not limited to, the following R branches R_{A}-R_{J}:

R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-

In each of these 10 examples, upper case “K” represents a L-lysine, and lower case “k” represents D-lysine. As indicated above, each H is independently L-histidine or D-histidine. In one aspect of these 10 examples, each H is L-histidine.

Specific HK polymers of the invention include, but are not limited to, HK polymers where each of R₁, R₂, R₃ and R₄ is the same and selected from R_{A}-R_{J}. These HK polymers are termed H2K4b, H3K4b, H3K(+H)4b, H3k(+H)4b, H-H3K(+H)4b, HH-H3K(+H)4b, H4K4b, H3K(1+H)4b, H3K(3+H)4b and H3K(1,3+H)4b, respectively.

In a second embodiment, the invention is directed to HK polyplexes comprising a HK polymer and a nucleic acid molecule, such as mRNA.

In a third embodiment, the invention is directed to HK associated lipid particles comprising a HK polymer, a nucleic acid molecule (such as mRNA), and a lipid moiety. Examples of lipid moieties include, but are not limited to, liposomes, micelles, fatty acyl groups, and cholesterol. These lipid moieties may be associated with the HK peptides by either ionic, covalent, and hydrophobic interactions. The liposome may be a cationic liposome such as, but not limited to, DOTAP (1,2-dioleoyl-3-(trimethylammonium) propane), DOSPER (1,3-dioleoyloxy-2-(6-carboxy-spermyl)-propylamid), DOTMA (N-[1-(2,3-dioleyloxy)-propyl]-N,N,N-trimethylammonium chloride), DC-cholesterol, DLinDMA (an ionizable 1,2-dilinoleyloxy-N,N-dimethyl-3-aminopropane), and an imidazole and/or histamine liposome. When a fatty acyl group or cholesterol (e.g., decanoyl, lauroyl, palmitoyl, stearoyl, arachidyl) serves as the lipid moiety, these may be conjugated with the HK polymers, and together with mRNA, form micelles.

In a fourth embodiment, the invention is directed to methods for inducing cellular uptake of a nucleic acid molecule, i.e. methods for transporting a nucleic acid molecule into a cell, and includes methods where the cell is cultured in vitro or present in an in vitro or ex vivo culture as well as methods were the cell is that of living animal, such as a human. Thus, the methods of the invention may be practiced in vitro, ex vivo or in vivo.

In one example of this embodiment, the invention is directed to methods for inducing cellular uptake of a nucleic acid molecule in vivo, comprising (i) mixing a nucleic acid molecule with a HK polymer under conditions permitting binding between the nucleic acid molecule and the HK polymer to form a HK polyplex, and (ii) administering the HK polyplex to a subject, where the HK polymer is a HK polymer as defined herein. As an example, the HK polymer may be a polymer in which each of R₁, R₂, R₃ and R₄ is the same and selected from R_{B}-R_{D}, defined above. The administration may be local (e.g., an injection) or systemic administration (e.g. IV administration).

In another example of this embodiment, the invention is directed to methods for inducing cellular uptake of a nucleic acid molecule in vivo, comprising (i) mixing a nucleic acid molecule with a HK polymer under conditions permitting binding between the nucleic acid molecule and the HK polymer to form a HK polyplex, (ii) mixing the HK polyplex with a lipid moiety under conditions permitting binding between the HK polyplex and the lipid moiety to form a HK associated lipid particle, and (iii) administering the HK associated lipid particle to a subject, where the HK polymer is a HK polymer as defined herein. As an example, the HK polymer may be a polymer in which each of R₁, R₂, R₃ and R₄ is the same and selected from R_{B}-R_{D}, defined above. The administration may be local (e.g., an injection) or systemic administration (e.g. IV administration).

In a further example of this embodiment, the invention is directed to methods for inducing cellular uptake of a nucleic acid molecule in vivo, comprising (i) mixing a HK polymer with a lipid moiety under conditions permitting binding between the lipid moiety and the HK polymer, (ii) mixing the HK polymer-lipid of (i) with a nucleic acid molecule under conditions permitting binding between the nucleic acid molecule and the HK polymer-lipid to form a HK associated lipid particle, and (iii) administering the HK associated lipid particle to a subject, where the HK polymer is a HK polymer as defined herein and wherein the lipid moiety is a lipid moiety as defined herein. As an example, the HK polymer may be a polymer in which each of R₁, R₂, R₃ and R₄ is the same and selected from R_{B}-R_{D}, defined above. The administration may be local (e.g., an injection) or systemic administration (e.g. IV administration).

In yet a further example of this embodiment, the invention is directed to methods for inducing cellular uptake of a nucleic acid molecule in vivo, comprising (i) mixing a lipid moiety with a nucleic acid molecule under conditions permitting between the nucleic acid molecule and the lipid moiety, (ii) mixing the nucleic acid molecule-lipid complex of (i) with a HK polymer under conditions permitting binding between the nucleic acid molecule-lipid complex and the HK polymer to form a HK associated lipid particle, and (iii) administering the HK associated lipid particle to a subject, where the HK polymer is a HK polymer as defined herein and wherein the lipid moiety is a lipid moiety as defined herein. As an example, the HK polymer may be a polymer in which each of R₁, R₂, R₃ and R₄ is the same and selected from R_{B}-R_{D}, defined above. The administration may be local (e.g., an injection) or systemic administration (e.g. IV administration).

In another example of this embodiment, the invention is directed to methods for inducing cellular uptake of a nucleic acid molecule in vitro, comprising (i) mixing a nucleic acid molecule with a HK polymer under conditions permitting binding between the nucleic acid molecule and the HK polymer to form a HK polyplex, and (ii) incubating the HK polyplex with a target cell under conditions permitting uptake by the cell of the HK polyplex, where the HK polymer is a HK polymer as defined herein. As an example, the HK polymer may be a polymer in which each of R₁, R₂, R₃ and R₄ is the same and selected from R_{B}-R_{D}, defined above.

In another example of this embodiment, the invention is directed to methods for inducing cellular uptake of a nucleic acid molecule in vitro, comprising (i) mixing a nucleic acid molecule with a HK polymer under conditions permitting binding between the nucleic acid molecule and the HK polymer to form a HK polyplex, (ii) mixing the HK polyplex with a lipid moiety under conditions permitting binding between the HK polyplex and the lipid moiety to form a HK associated lipid particle, and (iii) incubating the HK associated lipid particle with a target cell under conditions permitting uptake by the cell of the HK associated lipid particle, where the HK polymer is a HK polymer as defined herein. As an example, the HK polymer may be a polymer in which each of R₁, R₂, R₃ and R₄ is the same and selected from R_{B}-R_{D}, defined above.

In another example of this embodiment, the invention is directed to methods for inducing cellular uptake of a nucleic acid molecule in vitro, comprising (i) mixing a HK polymer with a lipid moiety under conditions permitting binding between the lipid moiety and the HK polymer, (ii) mixing the HK polymer-lipid of (i) with a nucleic acid molecule under conditions permitting binding between the nucleic acid molecule and the HK polymer-lipid to form a HK associated lipid particle, and (iii) incubating the HK associated lipid particle with a target cell under conditions permitting uptake by the cell of the HK associated lipid particle, where the HK polymer is a HK polymer as defined herein and wherein the lipid moiety is a lipid moiety as defined herein. As an example, the HK polymer may be a polymer in which each of R₁, R₂, R₃ and R₄ is the same and selected from R_{B}-R_{D}, defined above.

In another example of this embodiment, the invention is directed to methods for inducing cellular uptake of a nucleic acid molecule in vitro, comprising (i) mixing a lipid moiety with a nucleic acid molecule under conditions permitting binding between the nucleic acid molecule and the lipid moiety, (ii) mixing the nucleic acid molecule-lipid complex of (i) with a HK polymer under conditions permitting binding between the nucleic acid molecule-lipid complex and the HK polymer to form a HK associated lipid particle, and (iii) incubating the HK associated lipid particle with a target cell under conditions permitting uptake by the cell of the HK associated lipid particle, where the HK polymer is a HK polymer as defined herein and wherein the lipid moiety is a lipid moiety as defined herein. As an example, the HK polymer may be a polymer in which each of R₁, R₂, R₃ and R₄ is the same and selected from R_{B}-R_{D}, defined above.

In each of these examples, the ratio of the nucleic acid molecule to the HK polymer is from 2:1 to 1:12 (wt:wt).

In a further example of this embodiment, the invention is directed to methods for inducing cellular uptake of a nucleic acid molecule, comprising incubating a HK polyplex with a target cell under conditions permitting uptake by the cell of the HK polyplex, where the HK polyplex comprises a nucleic acid molecule and a HK polymer, and where the HK polymer is a HK polymer as defined herein. As an example, the HK polymer may be a polymer in which each of R₁, R₂, R₃ and R₄ is the same and selected from R_{B}-R_{D}, defined above. The method may be performed in vitro or in vivo.

In another example of this embodiment, the invention is directed to methods for inducing cellular uptake of a nucleic acid molecule, comprising incubating a HK associated lipid particle with a target cell under conditions permitting uptake by the cell of the HK associated lipid particle, where the HK associated lipid particle comprises a nucleic acid molecule, a HK polymer, and a lipid moiety, and where the HK polymer is a HK polymer as defined herein. As an example, the HK polymer may be a polymer in which each of R₁, R₂, R₃ and R₄ is the same and selected from R_{B}-R_{D}, defined above. The method may be performed in vitro or in vivo.

In each of the aspects and examples of this embodiment, the lipid moiety may be, but is not limited to, one or more of a liposome, micelle, fatty acyl group, and cholesterol. Suitable liposomes include, but are not limited to, DOTAP (1,2-dioleoyl-3-(trimethylammonium) propane), DOSPER (1,3-dioleoyloxy-2-(6-carboxy-spermyl)-propylamid), DOTMA (N-[1-(2,3-dioleyloxy)-propyl]-N,N,N-trimethylammonium chloride), DC-cholesterol, DLinDMA (an ionizable 1,2-dilinoleyloxy-N,N-dimethyl-3-aminopropane), or an imidazole and/or histamine liposome. Suitable fatty acyl groups include, but are not limited to, a decanoyl group, a lauroyl group, a palmitoyl group, a stearoyl group, and/or an arachidyl group. In one aspect, the HK associated lipid particle is in the form of a micelle with a fatty acyl group.

In the relevant embodiments and examples of the invention, the nucleic acid molecule may be, but is not limited to, mRNA.

In the relevant embodiments and examples of the invention, the cell may be, but is not limited to, an eukaryotic cell.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Comparison of H3K(+H)4b and H3K4b peptides as carriers of mRNA. To form the HK polyplexes, the mRNA (1 μg) was mixed with 3 different ratios of the HK (4, 8, 12 g) polymer for 30 minutes. The HK polyplexes were then added to the cells for 24 h before the luciferase activity was measured. ****, H3K(+H)4b vs H3K4b, P<0.0001.

FIG. 2. Titration of different HK peptide solutions. Solutions of polymers (5 mg/ml) (H2K4b, H3K4b, H3K(+H)4b, and H4K4b) were adjusted to pH 3 (initial volume—1 ml) and then 5 ml aliquots of 0.05 N NaOH were stepwise added and the pH measured.

FIG. 3. Gel retardation assay. After H3K(+H)4b or H3K4b carriers were mixed with mRNA at the various ratios shown in the figure (wt:wt; peptide:mRNA, mRNA was 1 μg in each) for 30 min, the HK polyplexes were loaded onto the gel (1% agarose). Electrophoresis was carried out at a constant voltage of 75 V for 30 min in TAE buffer and then stained with Sybr Gold as detailed herein. In first lane, RNA standard (New England Biolabls, B0363A) was loaded whereas in the second lane, the control, luciferase mRNA without a carrier was loaded into the well. In subsequent lanes, various ratios of mRNA:HK were loaded into wells.

FIG. 4. Heparin Displacement Assays. After HK polyplexes were formed (wt:wt; HK:mRNA; 4 μg:1 μg), different concentrations of heparin (0, 0.5, 1, 2, and 4 μg/ml) were incubated with these for 30 min. A. Gel Retardation. The HK polyplexes were loaded on the agarose gel (1%), and electrophoresis was carried out and stained with Sybr Gold. B. Fluorescent dye intercalation. After the HK polyplexes were formed and incubated with several concentrations of heparin, the Sybr Gold nucleic acid dye was incubated with the HK polyplexes for 5 mins. Fluorescence was then measured by a microplate fluorimeter (λex=497 nm, λem=520 nm) (SynergyMx, BioTek). *, P<0.05; ***, P<0.001; H3K4b or H2K vs. H3K(+H)4b.

FIG. 5. Fluorescent images of different HK polyplexes within acidic vesicles of MDA-MB-231 cells. A. Four h after transfection with labeled mRNA by H3K(+H)4b (upper) or H3K4b (lower) carriers, intracellular vesicles were labeled Lysotracker Green. The cells were then fixed, and the nuclei were stained with Hoesch 3342 dye. Images were obtained with a Nikon TE2000-S(Nikon JP) using a mercury lamp light source and filter sets delineated herein. (Green: acidic vesicles; Red: cynanine 5-labeled mRNA; Blue: nuclei labeled with Hoescht dye). The polymer mRNA polyplexes co-localized with the Lysotracker green, which accumulates within endocytic vesicles. Arrows indicate the irregularly shaped mRNA aggregates frequently observed with H3K4b carrier. B. Analysis of the amount of HK (H3K4b or H3K(+H)4b) polyplexes within acidic endosomal vesicles of MDA-MB-231 cells. Images of HK polyplexes labeled with cyanine5 (red emission) were imported into endosomal vesicles (green emission). The red/green ratios were measured on 20 intracellular acidic vesicles using the ImageJ software. The uptake of H3K(+H)4b polyplexes into acidic vesicles were significantly more than H3K4b polyplexes. Mann-Whitney Rank Sum Test: P<0.001, H3K(+H)4b vs. H3K4b polyplexes.

FIG. 6. Comparing H3K(+H)4b and other four-branched HK polymers for mRNA transfection. These HK polymers contain an additional histidine in the second motif H3k(+H)4b was the most effective peptide carrier of mRNA (H3k(+H)4b vs H3K(+4b); *, P<0.05).

FIG. 7. Comparison of H3K(+H)4b with branched polymers without an additional histidine in the second motif H3K(1+H)4b and H3K(3+H)4b peptides have an extra histidine in the first and third motifs, respectively. H3K(1,3+H)4b has two additional histidines, one in the first and the other third motif. Notably, the peptides (H3K(1+H)4b, H3K(3+H)4b, and H3K(1,3+H)4b) do not have an extra histidine in the second motif. Unlike H3K(+H)4b, the predominant repeating motif of H2K4b in its branch is -HHK. P<0.001, H3K(+H)4b vs. H2K4b or H3K(1,3+H)4b; P<0.0001, H3K(+H)4b vs H3K(1+H)4b or H3K(3+H)4b motifs.

FIGS. 8A and 8B. Comparison of mRNA transfection with DOTAP and several HK polymers. To form HK associated lipid particles, the mRNA (1 μg) was initially mixed with the HK polymer (4 μg) for 30 min and then with the DOTAP liposomes (1 μg) for 30 min. HK polyplexes were prepared as described previously. These complexes were then added to the cells for 24 h before the luciferase activity was measured *, P<0.05; **, P<0.01; ***, P<0.001; * P<0.0001.

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

Effective means for transferring nucleic acids into target cells are important tools, both in the basic research setting and in clinical applications. A diverse array of nucleic acid carriers is currently required because the effectiveness of a particular carrier depends on the characteristics of the nucleic acids that is being transfected [28-33]. For example, the large molecular weight branched polyethylenimine (PEI, 25 kDa) is an excellent carrier for plasmid DNA but not for mRNA. However, by decreasing the molecular weight of PEI to 2 kDa, it becomes a more effective carrier of mRNA [33].

Similarly, and in prior studies by the present inventors, the four-branched histidine-lysine (HK) peptide polymer H2K4b was shown to be a good carrier of large molecular weight DNA plasmids [27], but a poor carrier of relatively low molecular weight siRNA [34]. Further data from the same group showed that two histidine-rich peptides analogs of H2K4b, namely H3K4b and H3K(+H)4b, were effective carriers of siRNA [34, 35], although H3K(+H)4b appeared to be modestly more effective [45]. Moreover, the H3K4b carrier of siRNA induced cytokines to a significantly greater degree in vitro and in vivo than the H3K(+H)4b siRNA polyplexes [45].

The present inventors continued work on these histidine-lysine peptide polymers (“HK polymers” as commonly used herein) and surprisingly found, as reported herein, that some of the HK polymers are quite effective as mRNA carriers, and that they can be used, alone or in combination with liposomes, as effective means for direct delivery of mRNA into target cells. Similar to PEI and other carriers, initial results suggested HK polymers differ in their ability to carry and release nucleic acids. But because HK polymers can be made on a peptide synthesizer, their amino acid sequence can be easily varied, thus allowing fine control of the binding and release of mRNAs, as well as the stability of polyplexes comprising the HK polymers and mRNA [35-38].

### HK Polymers

The present invention is directed to branched polymers comprising four short peptide branches linked to a three-lysine amino acid core. Exemplary three-lysine core structures that may be used in the branched polymers of the invention are shown in Formula I and II. The peptide branches consist of histidine and lysine amino acids, in different configurations. The general structure of these histidine-lysine peptide polymers (HK polymers) is shown in Formula I and II, where R represents the peptide branches and K is the amino acid L-lysine.

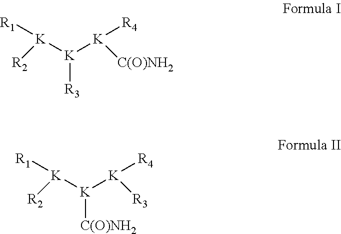

In the HK polymers of the invention represented by Formula I and II, each R is independently (i) KHₙKHₙKHₙKHₙK— (SEQ ID NO:1), (ii) HₙKHₙKHₙKHₙKHₙK— (SEQ ID NO:2), (iii) KHₙKHₙKHₙKHₙKHₙ— (SEQ ID NO:3), or (iv) HₙKHₙKHₙKHₙKHₙKHₙ— (SEQ ID NO:4), where H represents L-histidine or D-histidine, K represents L-lysine or D-lysine, and each n is independently an integer of between 0 and 4.

As suggested above, the R₁₋₄ branches may be the same or different in the HK polymers of the invention. Thus, the HK polymers include polymers where each of R₁, R₂, R₃, and R₄ are the same; where each of R₁, R₂, R₃, and R₄ are different; where R₁ is different and R₂, R₃ and R₄ are the same; where R₁, R₂ and R₃ are the same, and R₄ is different; where R₁ and R₂ are the same, and R₃ and R₄ are different; where R₁ and R₂ are different, and R₃ and R₄ are the same; where R₁ and R₂ are the same, and R₃ and R₄ are the same; where R₁ and R₃ are the same, and R₂ and R₄ are the same; where R₁ and R₃ are the same, and R₂ and R₄ are different; where R₁ and R₃ are different, and R₂ and R₄ are the same; where R₁ and R₄ are the same, and R₂ and R₃ are the same; where R₁ and R₄ are the same, and R₂ and R₃ are different; and where R₁ and R₄ are different, and R₂ and R₃ are the same. When a R branch is “different”, the amino acid sequence of that branch differs from each of the other R branches in the polymer.

Suitable R branches that may be used in the HK polymers of the invention include, but are not limited to, the following R branches R_{A}-R_{J}:

R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-

In each of these examples, upper case “K” represents a L-lysine, and lower case “k” represents D-lysine. As indicated above, each H is independently L-histidine or D-histidine. In one aspect of these 10 examples, each H is L-histidine.

Specific HK polymers of the invention include, but are not limited to, those shown in Table 1 where each of R₁, R₂, R₃, and R₄ is the same R branch shown in the table.

TABLE 1

| Polymer | Branch Sequence | Sequence Identifier |
|---|---|---|
| H2K4b | R = KHKHHKHHKHHKHHKHHKHK- | (SEQ ID NO: 5) |
| | 4 3 2 1 | |
| H3K4b | R = KHHHKHHHKHHHKHHHK- | (SEQ ID NO: 6) |
| H3K(+H)4b | R = KHHHKHHHKHHHHKHHHK- | (SEQ ID NO: 7) |
| H3k(+H)4b | R = kHHHkHHHkHHHHkHHHk- | (SEQ ID NO: 8) |
| H-H3K(+H)4b | R = HKHHHKHHHKHHHHKHHHK- | (SEQ ID NO: 9) |
| HH-H3K(+H)4b | R = HHKHHHKHHHKHHHHKHHHK- | (SEQ ID NO: 10) |
| H4K4b | R = KHHHHKHHHHKHHHHKHHHHK- | (SEQ ID NO: 11) |
| H3K(1 +H)4b | R = KHHHKHHHKHHHKHHHHK- | (SEQ ID NO: 12) |
| H3K(3 +H)4b | R = KHHHKHHHHKHHHKHHHK- | (SEQ ID NO: 13) |
| H3K(1,3 +H)4b | R = KHHHKHHHHKHHHKHHHHK- | (SEQ ID NO: 14) |

The numbers above the H3K4b peptide in Table 1 indicate the four repeating motifs present in each branch of the polymers. The lower case “k” in the sequence of H3k(+H)4b represents D-lysines in this construct. Extra histidine residues, in comparison to H3K4b, are underlined within the branch sequences. Nomenclature of the HK polymers is as follows: 1) for H3K4b, the dominant repeating sequence in the branches is -HHHK-, thus “H3K” is part of the name; the “4b” refers to the number of branches; 2) there are four -HHHK- motifs in each branch of H3K4b and analogues; the first -HHHK- motif (“1”) is closest to the lysine core; 3) H3K(+H)4b is an analogue of H3K4b in which one extra histidine is inserted in the second -HHHK- motif (motif 2) of H3K4b; 4) for H3K(1+H)4b and H3K(3+H)4b peptides, there is an extra histidine in the first (motif 1) and third (motif 3) motifs, respectively; 5) for H3K(1,3+H)4b, there are two extra histidines in both the first and the third motifs of the branches.

In each of the HK polymers of Table 1, the four R branches have identical amino acid sequences. However, the present invention encompasses HK polymers where 1, 2 or 3 of the branches have amino acid sequences that differ from R as defined in Table 1. These branches that may be different can each be independently selected from, for example, (i) KHₙKHₙKHₙKHₙK, (ii) HₙKHₙKHₙKHₙKHₙK, (iii) KHₙKHₙKHₙKHₙKHₙ, and (iv) HₙKHₙKHₙKHₙKHₙKHₙ as defined above. Alternatively, or in addition, these branches that may be different can each be independently selected from, for example, R_{A}-R_{J} as defined above.

### HK Polyplexes

When mixed with polynucleotides, such as mRNA, the HK polymers of the invention form spherical nanoparticles. The lysines of HK polymers are believed to interact electrostatically with the phosphates of nucleic acids, whereas the histidines have a number of roles including the assembly and disassembly of the nanoparticles and endosomal lysis [35].

As used herein, the terms “HK polyplex”, “HK polyplexes” and “polyplexes”, unless the context indicates otherwise, refers to the combination of a HK polymer and a polynucleotide molecule (e.g. mRNA). As shown in the examples discussed herein, the HK polymers of the invention can be used to transport nucleic acids, such as mRNA, into cells, in the form of HK polyplexes. For example, and as discussed in detail below, uptake of a H3K(+H)4b-mRNA polyplex by MDA-MB-231 cells, wherein the mRNA encoded luciferase, resulted in detectable levels of luciferase expression in the cells.

### HK Associated Lipid Particles

It is well-established that lipids, such as liposomes, can be used to facilitate transport of polynucleotide molecules, such as mRNA, into cells. As shown in the Examples discussed below, when liposomes, for example, are used in combination with HK polyplexes, synergistic results are achieved in terms of the amount of mRNA expression in cells, in comparison to use of either the liposome or HK polyplex alone.

Thus, the invention includes HK associated lipid particles. These lipid particles comprising a HK polymer, a nucleic acid molecule, such as mRNA, and a lipid moiety. Examples of suitable lipid moieties include, but are not limited to, liposomes, micelles, fatty acyl groups, and cholesterol. These lipid moieties may be associated with the HK peptides by either ionic, covalent, and hydrophobic interactions. The liposome may be a cationic liposome such as, but not limited to, DOTAP (1,2-dioleoyl-3-(trimethylammonium) propane), DOSPER (1,3-dioleoyloxy-2-(6-carboxy-spermyl)-propylamid), DOTMA (N-[1-(2,3-dioleyloxy)-propyl]-N,N,N-trimethylammonium chloride), DC-cholesterol, DLinDMA (an ionizable 1,2-dilinoleyloxy-N,N-dimethyl-3-aminopropane), and an imidazole and/or histamine liposome. When a fatty acyl group or cholesterol (e.g., a decanoyl group, a lauroyl group, apalmitoyl group, a stearoyl group, an arachidyl group) serves as the lipid moiety, these may be conjugated with the HK polymers, and together with mRNA, form micelles.

As used herein, the terms “HK associated lipid particle”, “HK associated lipid particles”, “lipid particle” and “lipid particles” refer to the combination of a HK polyplex(es) and a lipid moiety(ies) unless otherwise indicated by the context.

The HK associated lipid particles of the invention may be produced by (i) mixing the nucleic acids with the HK polymers, and then adding the lipid moieties for binding to the HK polyplexes, or (ii) mixing the HK polymers with the lipid moieties to form HK-polymer-lipids, and then adding the nucleic acids to be bound by the HK-polymer-lipids and thus forming the HK associated lipid particles of the invention, or (ii) mixing the lipid moieties with the nucleic acid molecules to form nucleic acid molecule-lipid complexes, and then adding the HK polymers to be bound by the nucleic acid molecule-lipid complexes and thus forming the HK associated lipid particles of the invention.

### Methods

As will be apparent from the description above, the HK polymers and HK associated lipid particles of the invention can be used to transport polynucleotide molecules into cells, i.e. induce cellular uptake of a nucleic acid molecule. Such methods can be used in vitro, ex vivo and in vivo. Thus, the present invention is also directed to methods for inducing cellular uptake of nucleic acid molecules into cells, whether the cells are in culture or in situ in a subject such as a human, a non-human primate, bird, horse, cow, goat, sheep, a companion animal, such as a dog, cat or rodent, or other mammal. The methods generally comprise (i) mixing a nucleic acid molecule with a HK polymer of the invention under conditions permitting binding between the nucleic acid molecule and the HK polymer to form a HK polyplex, and (ii) incubating the HK polyplex with a target cell under conditions permitting uptake by the cell of the HK polyplex. Alternative methods generally include (i) mixing a nucleic acid molecule with a HK polymer of the invention under conditions permitting binding between the nucleic acid molecule and the HK polymer to form a HK polyplex, (ii) mixing the HK polyplex with a lipid moiety under conditions permitting the formation of HK associated lipid particles, and (iii) incubating the HK associated lipid particles with a target cell under conditions permitting uptake by the cell of the HK associated lipid particles. Further alternative methods include (i) mixing a HK polymer with a lipid moiety under conditions permitting binding between the lipid moiety and the HK polymer, (ii) mixing the HK polymer-lipid of (i) with a nucleic acid molecule under conditions permitting binding between the nucleic acid molecule and the HK polymer-lipid to form a HK associated lipid particle, and (iii) incubating the HK associated lipid particle with a target cell under conditions permitting uptake by the cell of the HK associated lipid particle. Additional alternative methods include (i) mixing a lipid moiety with a nucleic acid molecule under conditions permitting between the nucleic acid molecule and the lipid moiety, (ii) mixing the nucleic acid molecule-lipid complex of (i) with a HK polymer under conditions permitting binding between the nucleic acid molecule-lipid complex and the HK polymer to form a HK associated lipid particle, and (iii) incubating the HK associated lipid particle with a target cell under conditions permitting uptake by the cell of the HK associated lipid particle.

The conditions permitting binding of the nucleic acid molecule by the HK polymer to form a HK polyplex generally comprise a room temperature mixture of nucleic acids and HK polymers in a cell culture media, such as the reduced serum media Opti-MEM (ThermoFisher Scientific), for a period of time, such as 15-60 minutes, to allow formation of the HK polyplexes. Suitable ratios of nucleic acid to HK polymer range from 50:1 to 1:50 (wt:wt). In particular aspects of the invention, the ratio ranges from 10:1 to 1:20, or from 2:1 to 1:12 (wt:wt). Specific ratios include 2:1, 1:1, 1:2, 1:3, 1:4, 1:5, 1:6, 1:7, 1:8, 1:9 and 1:10 (wt:wt). For in vitro transfections, medium (or buffers) which result in HK polyplexes or HK associated lipid particles with sizes ranging from 50 nm to 2 microns is desired. For in vivo transfections, medium which result in HK polyplexes or HK associated lipid particles with sizes between 50 nm and 300 nm is desired. HK polyplexes (or HK associated lipid particles) made in media (or buffers) with low ionic strength (e.g., water) usually have a reduced size whereas media with increased ionic strength (e.g., 0.15) have HK polyplexes (or HK associated lipid particles) with increased size. The composition of the HK peptide, the presence of cysteine with the HK peptide, and the amount of pegylation can affect the size of the HK polyplex or HK associated lipid particle in a specific media. The pH of the medium or buffer in which the HK polyplexes or HK associated lipid particles are made can range from pH 4 to 8.

The conditions permitting uptake by the cell of the HK polyplex or HK associated lipid particle generally comprises normal culture conditions for the cell being transfected. The normal culture conditions can include reduced concentrations of serum in the culture media, if normally present, for a portion of the time in which the HK polyplex HK associated lipid particle is being taken up by the cell.

The nucleic acid molecules that can be bound by the HK polymers of the invention, to form HK polyplexes and HK associated lipid particles includes individual nucleotides as well as polynucleotides. The nucleic acid molecules include DNA and RNA, such a genomic DNA, cDNA, mRNA, and siRNA.

The cells into which the HK polyplexes and HK associated lipid particles can be transfected include eukaryotic cells. When eukaryotic cells are the target, the cells may be those of a human, a non-human primate, bird, horse, cow, goat, sheep, a companion animal, such as a dog, cat or rodent, or other mammal.

The methods of the invention may be practiced in vitro, ex vivo or in vivo.

The amount of HK polyplexes and HK associated lipid particles that can be added to a cell culture, for in vitro or ex vivo methods, will depend on such factors as the identity of the cell, the culture conditions, the identity of the HK polymer and/or lipid moiety being used, and the identity of the nucleic acid being transported into a cell. However, when 24-well culture plates are used and the cells on the plate are at a 60-80% confluence, between about 0.1 and 100 g of HK polyplexes or HK associated lipid particles may be cultured with the cells.

The amount of HK polyplexes and HK associated lipid particles that can be administered to a subject, for in vivo methods, will depend on such factors as the weight and medical condition of the subject, the identity of the HK polymer and/or lipid moiety being used, and the identity of the nucleic acid being transported into a cell. However, between about 0.1 and 100 μg of HK polyplexes or HK associated lipid particles per kg of body weight of the subject may be administered.

The HK polyplexes and HK associated lipid particles may be formulated, for example, for oral, sublingual, intranasal, intraocular, rectal, transdermal, mucosal, pulmonary, topical or parenteral administration. Parenteral modes of administration, whether local or systemic, include without limitation intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

### III. Examples

Peptides. The HK polymers were synthesized on a Ranin Voyager synthesizer (Tucson, AZ) by the biopolymer core facility at the University of Maryland or by Genscript (Piscataway, NJ) as previously described [34, 39]. To ensure a purity of 90% or greater [39], peptides were analyzed by high-performance liquid chromatography (Beckman Coulter, Fullerton, CA, USA) with System Gold operating software, using a Dynamax 21.4×250 mm C-18 reversed-phase preparative column with a binary solvent system. Further analyses of the peptides were done by ESI mass spectroscopy (LCMS-2020, SHIMADZU Corporation, Kyoto, JP). The sequences of the peptides that make up some of the HK polymers of the invention are shown in Table 1.

In vitro mRNA Transfection. Several HK polymers were examined for their ability to carry a luciferase-expressing mRNA (CleanCap Firefly Luciferase mRNA, Trilink Biotechnologies, Inc, San Diego, CA) into MDA-MB-231 cells (America Type Tissue Culture, Manassas, VA). In brief, 3×10⁴ cells were plated into a 24-well plate containing 500 μl of DMEM and 10% fetal bovine serum (ThermoFisher Scientific, Waltham, MA). After 24 h, when the cells were 60 to 80% confluent, the media in each well was changed to Opti-MEM (ThermoFisher Scientific). To prepare HK polyplexes, mRNA (1 μg) in 50 μl of Opti-MEM was briefly mixed well with one of the HK polymers (4 to 12 μg) and maintained at room temperature for 30 min. This polyplex was then added dropwise to the cells. After four h, the Opti-MEM media was removed and replaced with 0.5 ml of DMEM/10% serum (0.5 ml). Twenty-four hours later, the cells were lysed, and the luciferase activity (Promega Corporation, Madison, WI) was measured [27].

Transfection with HK associated lipid particles was done similarly as above with few exceptions. In brief, the HK polymer was mixed initially with mRNA at various ratios for 30 min in Opti-MEM. This was followed by adding the DOTAP cationic liposome (1,2-dioleoyl-3-trimethylam-monium-propane; 1 kg; Roche, Basel, CH) for 30 additional min. The Opti-MEM mixture (50 μl) was then added dropwise to the cells.

Acid-Base Titration. After polymer solutions (e.g. containing one or more of H2K4b, H3K4b, H3K(+H)4b, and H4K4b) were adjusted to pH 3.0 (5 mg/mil; initial volume—1 mil), aliquots (5 μl) of NaOH (0.05 N) were stepwise added with the pH measured (FiveEasy™ meter; InLab Solids Pro-ISM pH electrode, Mettler Toledo, Columbus, OH). Titration was stopped at about pH 9.0.

Cell Viability Assay. MDA-MB-231 cells were seeded at 5.0×10⁴/well in a 24-tissue culture plate and incubated overnight in DMEM supplemented with 10% FBS serum. The media was then changed to Opti-MEM and the cells were treated with either the HK polymer (4 μg) or the HK polyplex (4 μg HK; 1 μg mRNA) for 5 h. After the media was changed to DMEM/10% FBS for 19 h, the cell viability was measured using the trypan blue cell exclusion assay (Trypan Blue solution, 0.4%, Sigma-Aldrich, St. Louis, MO) [40].

Gel Retardation Assay. Various amounts of HK polymers were mixed with 1 kg of mRNA and incubated for 30 min at room temperature. Specifically, the following HK polymer/mRNA ratios (w/w) were prepared in water: 1/2; 1/1; 2/1, 4/1, 8/1. After 30 min, the HK polyplex was loaded onto the gel (1% agarose, Sigma-Aldrich; 10× BlueJuice Gel loading buffer, ThermoFisher Scientific), electrophoresis was then carried out at a constant voltage of 75 V for 30 min in TAE buffer (Quality Biologicals, Gaithersburg, MD). The mRNA was stained with Sybr Gold Nucleic Acid dye (SG, 1×) (ThermoFisher Scientific) for 30 min before exposure to the UV imager (ChemiDoc Touch, BIO-RAD, Hercules, CA).

Heparin Displacement Assays. Heparin displacement assays of HK polyplexes were done with the dye intercalation assay and with gel electrophoresis. A fluorescent assay assessed polyplexes of HK polymer and mRNA (4:1 wt/wt ratio; polymer:mRNA) formed in RNAse/DNAase free water (Corning, Manassas, VA). HK polyplexes were prepared as described previously, followed by the addition of diluted Syber Gold. For detection, working dilutions of the polyplexes (1/5 of volume), water (3/5) and Sybr Gold dye (1/5, 0.2×) were incubated for 5 minutes, and fluorescence was measured by a fluorimeter (Ex=497 nm, Em=520 nm) (SynergyMx, BioTek, Winooski, VT). The control sample was prepared with the same amount of mRNA, water, and Sybr Gold dye. For the heparin displacement, instead of water, heparin salt (Sigma-Aldrich, St. Louis, MO) solutions at different concentrations (0.5, 1, 1.5, 2, 3 μg/μl) were used, and the HK polyplexes were incubated at 37° C. for 30 min before addition of Sybr Gold.

Displacement of mRNA from HK polyplexes with heparin was also done with gel electrophoresis. After HK polyplexes were formed, different concentrations of heparin (0.5, 1.0, 1.5, 2.0, 3.0 μg/μl; volume 20 μl) were incubated with these at 37° C. for 30 min. The polyplexes were then loaded on the agarose gel (1%; 10× BlueJuice Gel loading buffer), and electrophoresis was carried out and stained with SG as described above. Images were acquired by UV imager (ChemiDoc Touch, BIO-RAD).

In vitro uptake of HK polyplexes by fluorescence microscopy. With the mRNA labeled with Cy5, HK polyplexes at 4:1 ratio (HK:mRNA) were prepared as described in the in vitro transfection section above. The labeled HK polyplexes were incubated with MDA-MB-231 cells for four h in Opti-MEM. After the cells were washed with phosphate buffer saline (PBS, Quality Biologicals, Gaithersburg, MD), they were incubated for 30 min with LysoTracker Green DND-26 (Cell Signaling Technology, Inc., Danvers, MA), a dye that stains acidic endosomes and lysosomes. Then after the cells were washed twice with PBS twice and once with 1% Triton-X, they were fixed (4% formalin/1% glutaraldehyde), and the nuclei were stained with chromatin dye Hoechst 33342 (Invitrogen, Carlsbad, CA). Images were obtained with a Nikon TE2000-S(Nikon, Tokyo, JP) with a mercury lamp light source using the following filter sets: Ex-357(20)/Em-460(60) (Hoechst); Ex-480(30)/Em-535(45)-Lysotracker green DND-26; Ex-620(50)/Em-690(50)—(Cy5-labelled-mRNA). Red/green ratios were measured on 20 intracellular acidic vesicles (one per cell) using the ImageJ software (version 1.52v) [41].

In vitro uptake of HK polyplexes by flow cytometry. Intracellular uptake of HK polyplex in MDA-MB-231 was measured by flow cytometry. Twenty-four hours before the treatment, cells were plated in a 24-well plate. The HK polyplex was formed in Opti-MEM at the ratio of 4:1 (HK:mRNA), at room temperature for 30 minutes. Then, H3K(+H)4b or H3K4b mRNA polyplexes (cyanine 5′-labeled mRNA, Trilink Biotechnologies) were added to the cell culture medium. At several time points (1, 2, and 4 hours), transfected cells were harvested, fixed with 4% formalin/1% glutaraldehyde, and resuspended in PBS buffer for analysis. Results from the fluorescently labeled MDA-MB-231 cells were then acquired using Cytoflex (Beckman Coulter) and analyzed using CytExpert software (Version 2.3.0.84) on the flow cytometer.

Stability of HK polyplexes to enzymatic degradation. After preparation of the H3K4b or H3k(+H)4b mRNA polyplexes (wt:wt; HK (0.5, 1, or 4 μg):mRNA (1 μg)), these polyplexes were incubated with trypsin (0.025%) for 30 or 60 min. The HK polyplexes were then loaded on a 1% agarose gel and electrophoresis was carried out at 75 V for 30 min in TAE buffer. The gel was stained in a TAE buffer containing ethidium bromide (1 μg/ml) for 10 min.

Particle size, polydispersity index (PDI), and zeta potential. The size, PDI, and zeta potential were determined with the Zetasizer (Malvern, Westborough, Mass.) and analyzed with software provided by the instrument manufacturer (Zetasizer software, version 6.2). Using dynamic light scattering at a 90° angle, the size of the particles were reported as the Z-average diameter from the intensity-weighted size distribution. Prior to the measurements, the samples were equilibrated to 25° C. for 2 min. Each measurement had at least ten sub-runs under the automatic mode of the software. The particle size, PDI, and zeta potential data point represent the mean±SD of three measurements. After mixing HK peptides (4 μg) and mRNA (1 μg) in 100 μl of defined media (Opti-MEM, water, or DMEM/8% FBS) for 30 min, 100 μl of additional defined media was added to the polyplex solution (total volume 200 μl) to measure the size and PDI. To determine the zeta potential, 800 μl more of the media was added (total volume 1000 μl), mixed gently, and then added to the disposable zeta cell.

Statistical Analysis. Results, reported as mean±standard deviation (+SD), represent three separate data measurements unless otherwise indicated. Except where stated, results were analyzed using a two-tailed t-test with a single asterisk representing P<0.05, a double asterisk, P<0.01, a triple asterisk representing P<0.001, and a quadruple asterisk representing P<0.0001(SigmaPlot, San Jose, CA).

### Results

H3K(+H)4b is a Significantly Better Carrier than H3K4b

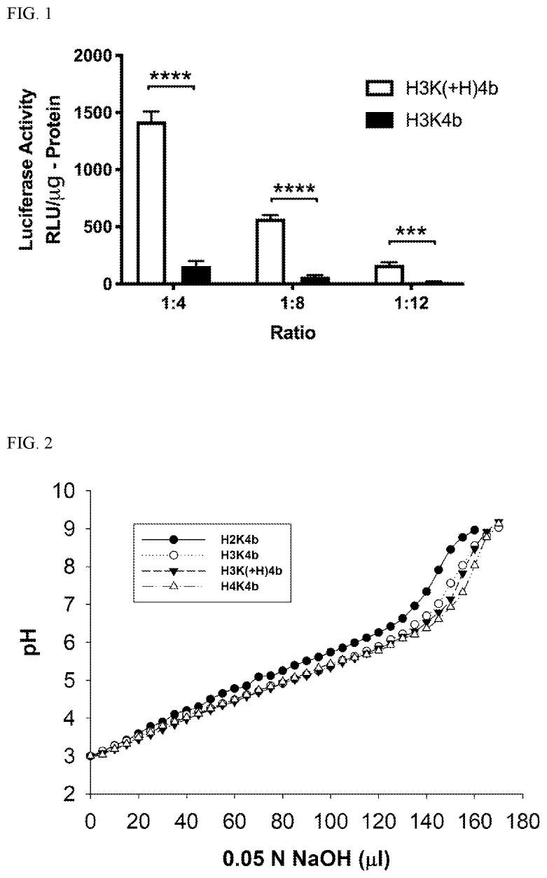

Both H3K4b and H3K(+H)4b have shown promise as carriers of nucleic acids in vitro [34, 42]. Despite these previous findings, H3K(+H)4b was markedly better as a carrier of mRNA compared to its close H3K4b analogue (FIG. 1; H3K(+H)4b—left columns; H3K4b—right columns). At the 4:1 ratio (HK:mRNA; wt:wt), luciferase expression was 10-fold greater with the H3K(+H)4b than with the H3K4b peptide in MDA-MB-231 cells. Moreover, the buffering capacity does not seem to be an essential factor in their transfection differences since the percent of histidines (by weight) in H3K4b and H3K(+H)4b is 68.9 and 70.6%, respectively. Furthermore, the pH titration curves of H3K4b and H3K(+H)4b HK polymers corroborated minimal differences in their buffering profile (FIG. 2).

At the HK peptide: mRNA ratio used in these initial experiments, neither polyplex showed cytotoxicity toward MDA-MB-231 cells. After the medium was changed to Opti-MEM in cell cultures of the MDA-MB-231 cells used in the experiment described above, either the HK polymer (4 μg) or the HK polyplex (4 μg HK; 1 μg mRNA) was added dropwise to the cells and incubated for 5 h. The media was then changed to DMEM/10% FBS for 19 h and cell viability was determined using the trypan cell exclusion assay. The results are shown in Table 2.

TABLE 2
Trypan Blue Exclusion Assay

| Treatment | % Viability |
|---|---|
| Untreated Cells | 97.6 |
| H3K4b | 95.6 |
| H3K4b + mRNA | 95.4 |
| H3(+H)K4b | 96.3 |
| H3(+H)K4b + mRNA | 94.3 |

### Gel Retardation and Heparin Displacement Assays Indicate Differences in Stability

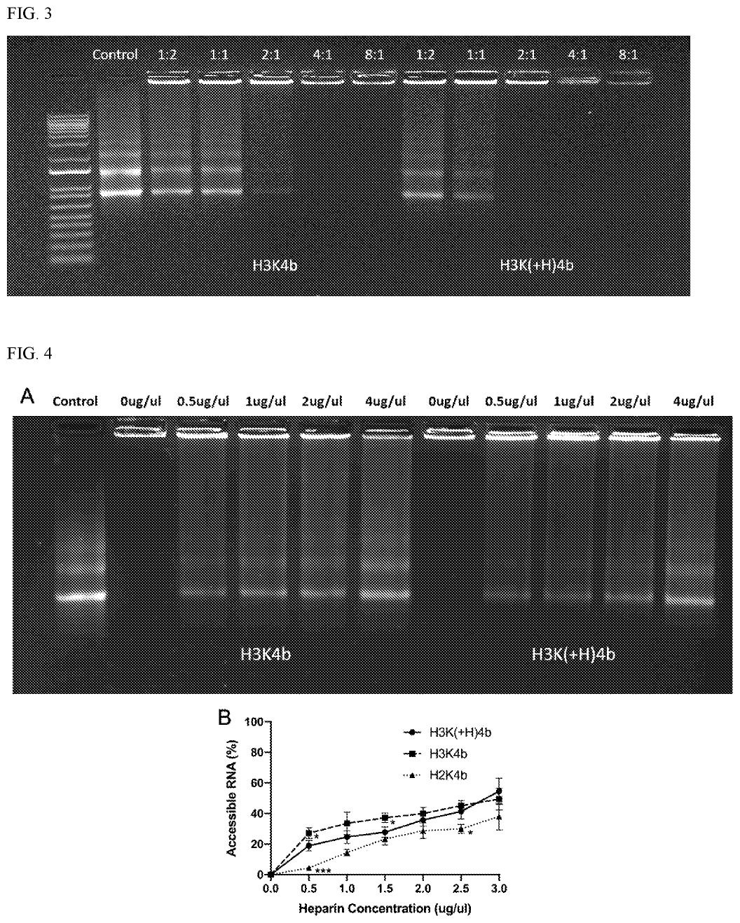

Next, gel retardation assays were performed and the results showed the effect of polypeptides in different weight ratios of mRNA and peptide (FIG. 3). The results indicate that the electrophoretic mobility of mRNA was delayed by the HK polymers. The retardation effect increased with higher peptide to mRNA weight ratios. mRNA was markedly less retarded at the 1:2 and 1:1 ratios (wt:wt; peptide:mRNA) of H3K(+H)4b compared to the same ratios of H3K4b. With 2:1 and 4:1 ratios, the mRNA was completely entrapped by the H3K(+H)4b polyplex, whereas only with the 4:1 ratio was the mRNA completely retarded by the H3K4b polyplex. These results suggest that the H3K(+H)4b polymer forms a more stable polyplex, and this may play a role in the reason why H3K(+H)4b is more effective as a carrier compared to H3K4b.

Further confirmation that the H3K(+H)4b peptide binds more tightly to the mRNA was demonstrated with a heparin-binding assay (FIG. 4, A,B). Particularly at the lower concentrations of heparin, mRNA was released by the H3K4b polymer more readily than the H3K(+H)4b polymer. These data, together with the size of polyplexes in different media discussed below, suggest that H3K(+H)4b polyplexes may be more stable than the H3K4b polyplexes. Nevertheless, if a peptide such as H2K4b forms a polyplex that is too stable, this may also reduce mRNA transfection (see FIG. 4B). Interestingly, other HK polyplexes that showed effective mRNA transfection had similar stabilities when exposed to heparin as the H3K(+H)4b polyplexes (data not shown). Moreover, the H3K(3+H)4b polyplex, which was an ineffective carrier of mRNA, had a similar stability as the H3K4b polyplex.

Because different stabilities were observed between the H3K4b and H3K(+H)4b polyplexes, whether the sizes of these polyplexes varied based on the media in which they were prepared was investigated. Both H3K4b and H3K(+H)4b polyplexes had a similar size and PDI in water, but when they were prepared in media with higher salt and/or serum, H3K4b polyplexes were markedly larger. H3K(+H)4b and H3K4b peptides (4 μg) in complex with mRNA (1 μg) were mixed with either Opti-MEM, water, or DMEM/8% FBS (100 ml). After 1 h, the size, PDI, and zeta potential were measured. The results are shown in Table 3.

TABLE 3
The size and zeta potential of the polyplexes in different media

| | | |
|---|---|---|
| Opti-MEM | Water | DMEM/8% FBS |

Size(nm)

| | | | |
|---|---|---|---|
| H3K(+H)4b | 1004 ± 61.1 | 234.9 ± 2.7 | 289 ± 38.2 |
| H3K4b | 2030.7 ± 117.3 | 199.9 ± 0.8 | 578 ± 80.5 |

Zeta Potential(mV)

| | | | |
|---|---|---|---|
| H3K(+H)4b | 2.92 ± 1.83 | 20.77 ± 1.12 | −12.7 ± 1.04 |
| H3K4b | −4.20 ± 2.78 | 16.47 ± 0.74 | −12.1 ± 2.22 |

PDI

| | | | |
|---|---|---|---|
| H3K(+H)4b | 0.362 | 0.212 | 0.307 |
| H3K4b | 0.466 | 0.171 | 0.421 |

### Increased Intracellular Localization of H3K(+H)4b Polyplexes Compared to H3K4b

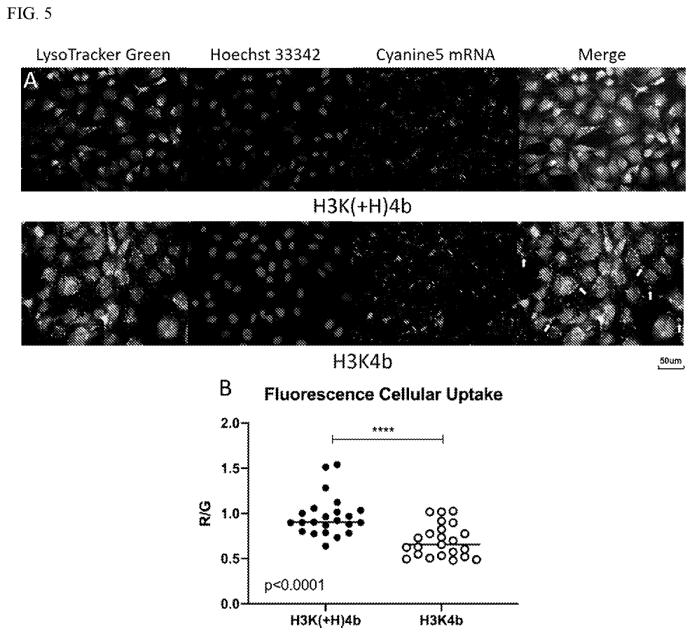

With the mRNA labeled with cyanine-5, the uptake of H3K4b and H3K(+H)4b polyplexes into MDA-MB-231 cells was compared using flow cytometry. At different time points (1, 2, and 4 h), the H3K(+H)4b polyplexes were imported into the cells more efficiently than H3K4b polyplexes (data not shown). Similar to these results, fluorescent microscopy indicated that H3K(+H)4b polyplexes localized within the acidic endosomal vesicles significantly more than H3K4b polyplexes (H3K4b vs. H3K(+H)4b, P<0.001) (FIGS. 5A and B). Interestingly, irregularly-shaped H3K4b polyplexes, which did not overlap endocytic vesicles, were likely extracellular and were not observed with H3K(+H)4b polyplexes (FIG. 5A).

Transfection of mRNA with HK Carriers with Extra Histidine in the Second Motif is Essential for mRNA Transfection

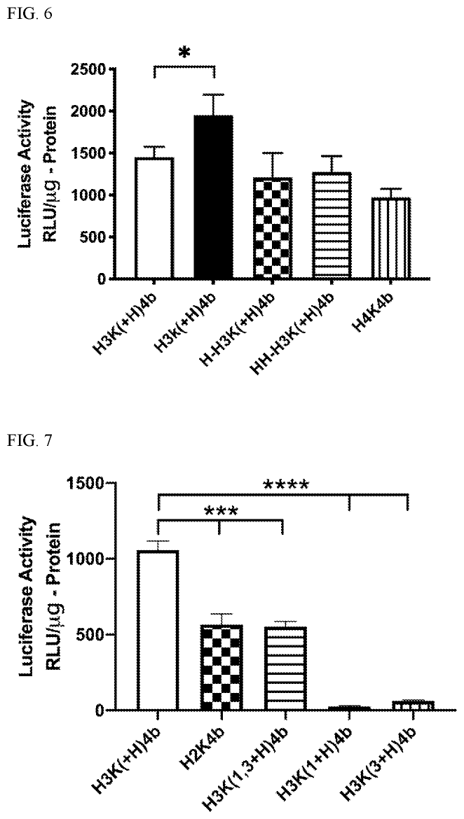

All the HK polymers with an extra histidine in the second -HHHK motif of the branches were effective carriers of mRNA (FIG. 6). Of these peptides, H3k(+H)4b was determined to be the optimal carrier of mRNA (H3k(+H)4b vs. H3K(+H)4b, P<0.05). With this peptide, the L-lysines were replaced with D-lysines, and enhanced stability of resulting polyplexes may be the reason why this polymer was a better carrier than H3K(+H)4b. This was based on prior antimicrobial studies in which replacement of L-lysines with D-lysines suggested that H3k4b (a close analog of H3k(+H)4b) was more stable to enzymatic degradation, had greater antimicrobial activity, and had no observed cytotoxicity to human cells [43]. Exposure to trypsin provided further support that the H3k(+H)4b mRNA polyplexes had enhanced stability to enzymatic degradations compared to H3K(+H)4b polyplexes (data not shown).

Interestingly, additional histidines in locations other than the second motif do not appear to be a critical factor in enhancing mRNA transfection (FIG. 6). For example, H4K4b with twelve more histidines per peptide than H3K(+H)4b did not enhance mRNA transfection. Notably, the percent of histidine content in H3K(+H)4b and H4K4b peptides was about 70.5 and 75%, respectively, and their similar buffering capacity was corroborated with the pH titration profile (FIG. 2). Moreover, H-H3K(+H)4b and HH-H3K(+H)4b peptides with additional histidines did not improve mRNA transfection more than H3K(+H)4b (FIG. 6). Nevertheless, these three peptides (H4K4b, H-H3K(+H)4b, and HH-H3K(+H)4b) with a histidine in the second motif were effective carriers of mRNA, similar to the H3K(+H)4b carrier.

When the branched HK polymers with a predominant pattern of -HHK- did not have an additional histidine in the second motif, mRNA transfection was markedly reduced (FIG. 7, Table 1). For example, although the H3K(1,3+H)4b peptide has an additional histidine in its first and third motif (compared to H3K4b, Table 1), it does not have the extra histidine in the second motif. H3K(1,3+H)4b was about 2.5-fold less effective in transfecting mRNA compared to H3K(+H)4b (P<0.001). Moreover, although H3K(1,3+H)4b and H-H3K(+H)4b had the same number of histidines and lysines per branch, H-H3K(+H)4b was markedly more effective as a carrier of mRNA (FIG. 6, 7). In contrast to the H3K(1,3+H)4b peptide, the H-H3K(+H)4b peptide has an extra histidine in the second motif.

Similar to H3K4b and H3K(1,3+H)4b polymers, two other peptide carriers (H3K(1+H)4b and H3K(3+H)4b) that did not have an additional histidine in the second motif were poor carriers of mRNA (FIG. 7, Table 1, Table 4). H3K(1+H)4b and H3K(3+H)4b have an extra histidine in first and third motif, respectively. Thus, the location of the histidines in the branches appears to be important.

TABLE 4
Transfection of mRNA with four-branched HK Polymers

| Polymers | Ratio(wt:wt; mRNA:Polymer) | RLU/μg-Protein |
|---|---|---|
| H3K(+H)4b | 1:4 | 1532.9 ± 122.9 |
| | 1:8 | 1656.3 ± 202.5 |
| | 1:12 | 1033.4 ± 197 |
| H3k(+H)4b | 1:4 | 1851.6 ± 138.3 |
| | 1:8 | 1787.2 ± 195.2 |
| | 1:12 | 1982.3 ± 210.7 |
| H3K4b | 1:4 | 156.8 ± 41.8 |
| | 1:8 | 62.1 ± 13.2 |
| | 1:12 | 18.1 ± 4.0 |
| H3K(₃₊H)4b | 1:4 | 61.7 ± 5.7 |
| | 1:8 | 68.7 ± 3.1 |
| | 1:12 | 59.0 ± 7.5 |
| H3K(₁₊H)4b | 1:4 | 24.3 ± 4.5 |
| | 1:8 | 15.0 ± 3.6 |
| | 1:12 | 7.3 ± 2.5 |
| H-H3K(+H)4b | 1:4 | 1107.5 ± 140.4 |
| | 1:8 | 874.6 ± 65.2 |
| | 1:12 | 676.4 ± 25.7 |
| HH-H3K(+H)4b | 1:4 | 1101.9 ± 106.6 |
| | 1:8 | 832.2 ± 75.3 |
| | 1:12 | 739.8 ± 105.4 |
| H4K4b | 1:4 | 896.4 ± 112.6 |
| | 1:8 | 821.8 ± 115.6 |
| | 1:12 | 522.4 ± 69.2 |
| H3(1,3 + H)K4b | 1:4 | 518.3 ± 134.7 |
| | 1:8 | 427.7 ± 18.1 |
| | 1:12 | 378 ± 5.2 |
| H2K4b | 1:4 | 546.7 ± 70.1 |
| | 1:8 | 132.3 ± 58.5 |
| | 1:12 | 194.7 ± 18.4 |

To obtain the data in Table 4, luciferase-expressing mRNA (1 μg) in 50 μl of Opti-MEM was briefly mixed with one of the HK polymers (4, 8, or 12 μg) and maintained at room temperature for 30 min. The resulting polyplexes were added dropwise to the MDA-NM-231 cells and after four h, the Opti-MEM media was removed and replaced with DMEM/10% serum. Twenty-four hours later, the cells were lysed, and the luciferase activity was measured

Although the data for FIG. 6, 7 was obtained with the 4:1 ratio (wt:wt, HK:mRNA), analogous results were generally found at 8:1 and 12:1 ratios during the initial screening (Table 4). An exception was with H2K4b, a branched peptide with a predominant sequence of -HHK. Although H2K4b carrier resulted in a similar yet low transfection of mRNA as H3K(1,3+H)4b at the 4:1 ratio (FIG. 7), transfection with H2K4b at the 8:1 and 12:1 ratios was further reduced (Table 4). Compared to other branched HK polymers in this study, H2K4b had the highest percentage of lysines.

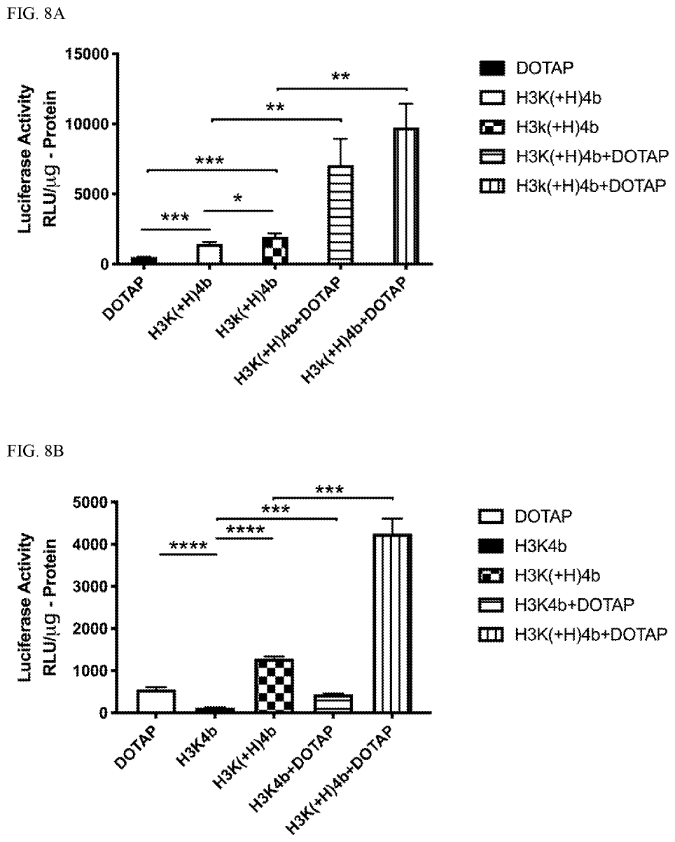

It is known both that HK polymers and cationic liposomes (i.e., DOTAP) significantly and independently increase transfection with plasmids [44]. Consequently, whether these liposomes together with HK polymers enhanced mRNA transfection was investigated. Notably, the H3K(+H)4b and H3k(+H)4b carriers were significantly better carriers of mRNA than the DOTAP liposomes (P<0.001) (FIG. 8A, 8B). It was determined that the combination of H3K(+H)4b and DOTAP liposomes was synergistic in the ability to carry mRNA into MDA-MB-231 cells (FIG. 8B). The combination was about 3-fold and 8-fold more effective as carriers of mRNA than the polymer alone and the liposome carrier, respectively (H3K(+H)4b/liposomes vs. liposomes or H3K(+H)4b, P<0.0001). Notably, not all HK peptides demonstrated improved activity with DOTAP liposomes. The combination of H3K4b and DOTAP carriers was less effective than the DOTAP liposomes as carriers of luciferase mRNA (P<0.05) (FIG. 8B).

As stated previously, the D-isomer, H3k(+H)4b, was the most effective polymeric carrier (FIG. 6). The D-isomer/liposome carrier of mRNA was nearly 4-fold and 10-fold more effective than the H3k(+H)4b alone and liposome carrier, respectively (FIG. 8A). Although the D-isomer H3k(+H)4b/liposome combination was modestly more effective than the L-isomer H3K(+H)4b/liposome combination, this comparison was not statistically different.

As demonstrated in the examples and discussed above, it has been shown herein that the H3K(+H)4b-mRNA polyplex was about 10-fold more efficient in expressing luciferase in MDA-MB-231 cells compared to H3K4b-mRNA polyplex. Thus, the addition of a single histidine to the second motif of H3K4b enhanced mRNA transfection. However, the addition of histidines to the branched HK polymers did not necessarily improve the efficacy of the carrier in transporting mRNA. For instance, the addition of two histidines to the N-terminal ends of the branches of H3K(+H)4b did not increase luciferase expression. Nevertheless, all five of the branched HK polymers with the extra histidine in the second motif were effective carriers of mRNA.

At least part of the transfection differences between H3K(+H)4b and H3K4b particles appear to be due to the structural and biophysical differences. As gel retardation and heparin displacement assays demonstrated, the H3K(+H)4b polyplexes showed greater stability than H3K4b polyplexes. Moreover, although these two HK polyplexes have a similar size when formed in water, the polyplexes of H3K4b were markedly larger when formed in Opti-MEM or serum. The smaller and more stable particles formed by H3K(+H)4b could favor enhanced cellular uptake via endocytosis and contribute to enhanced intracellular mRNA delivery.

The enhanced stability of H3K(+H)4b polyplexes was further illustrated by the fluorescence images of the nanoparticles within the cell. Whereas the fluorescence of H3K(+H)4b-mRNA overlapped the acidic endosomal vesicles to a significant degree, the fluorescence of H3K4b-mRNA nanoparticles overlapped to a much lesser degree. Moreover, the irregular-shaped extracellular H3K4b polyplexes, which did not overlap with endosomes, were not observed with H3K(+H)4b polyplexes, and the results suggest that decreased uptake may be a primary reason of the inefficiency of H3K4b carrier. In addition to the reduced uptake by H3K4b polyplexes, the increased release of mRNA from H3K4b polyplexes may play a role in the reduced transfection compared to H3K(+H)4b polyplexes.

The combination of DOTAP and H3K(+H)4b carriers were found to be synergistic in their ability to carry mRNA into cells.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Zohra F T, Maitani Y, Akaike T mRNA delivery through fibronectin associated liposome-apatite particles: a new approach for enhanced mRNA transfection to mammalian cell. Biol Pharm Bull 2012; 35: 111-115.
2. Phua K K, Leong K W, Nair S K Transfection efficiency and transgene expression kinetics of mRNA delivered in naked and nanoparticle format. J Control Release 2013; 166: 227-233.
3. Pardi N, Tuyishime S, Muramatsu H, et al. Expression kinetics of nucleoside-modified mRNA delivered in lipid nanoparticles to mice by various routes. J Control Release 2015; 217: 345-351.
4. Kowalski P S, Rudra A, Miao L, et al. Delivering the Messenger: Advances in Technologies for Therapeutic mRNA Delivery. Mol Ther 2019; 27: 710-728.
5. Freitag F, Wagner E Optimizing synthetic nucleic acid and protein nanocarriers: The chemical evolution approach. Adv Drug Deliv Rev 2020. doi: 10.1016/j.addr.2020.03.005.
6. Jarzebinska A, Pasewald T, Lambrecht J, et al. A Single Methylene Group in Oligoalkylamine-Based Cationic Polymers and Lipids Promotes Enhanced mRNA Delivery. Angew Chem Int Ed Engl 2016; 55: 9591-9595.
7. McKinlay C J, Benner N L, Haabeth O A, et al. Enhanced mRNA delivery into lymphocytes enabled by lipid-varied libraries of charge-altering releasable transporters. Proc Natl Acad Sci USA 2018; 115: E5859-E5866. doi: 10.1073/pnas.1805358115
8. McKinlay C J, Vargas J R, Blake T R, et al. Charge-altering releasable transporters (CARTs) for the delivery and release of mRNA in living animals. Proc Natl Acad Sci USA 2017; 114: E448-E456. doi: 10.1073/pnas.1614193114.
9. Wang F, Xiao W, Elbahnasawy M A, et al. Optimization of the Linker Length of Mannose-Cholesterol Conjugates for Enhanced mRNA Delivery to Dendritic Cells by Liposomes. Front Pharmacol 2018; 9: 980. doi: 10.3389/fphar.2018.00980.
10. Kabilova T O, Shmendel E V, Gladkikh D V, et al. Targeted delivery of nucleic acids into xenograft tumors mediated by novel folate-equipped liposomes. Eur J Pharm Biopharm 2018; 123: 59-70.
11. Brito L A, Chan M, Shaw C A, et al. A cationic nanoemulsion for the delivery of next-generation RNA vaccines. Mol Ther 2014; 22: 2118-2129.
12. Badieyan Z S, Pasewald T, Mykhaylyk O, et al. Efficient ex vivo delivery of chemically modified messenger RNA using lipofection and magnetofection. Biochem Biophys Res Commun 2017; 482: 796-801.
13. Zohra F T, Chowdhury E H, Akaike T High performance mRNA transfection through carbonate apatite-cationic liposome conjugates. Biomaterials 2009; 30: 4006-4013.
14. Uchida H, Itaka K, Nomoto T, et al. Modulated protonation of side chain aminoethylene repeats in N-substituted polyaspartamides promotes mRNA transfection. J Am Chem Soc 2014; 136: 12396-12405.
15. Crowley S T, Poliskey J A, Baumhover N J, et al. Efficient expression of stabilized mRNA PEG-peptide polyplexes in liver. Gene Ther 2015; 22: 993-999.
16. Nuhn L, Kaps L, Diken M, et al. Reductive Decationizable Block Copolymers for Stimuli-Responsive mRNA Delivery. Macromol Rapid Commun 2016; 37: 924-933.
17. Chen Q, Qi R, Chen X, et al. A Targeted and Stable Polymeric Nanoformulation Enhances Systemic Delivery of mRNA to Tumors. Mol Ther 2017; 25: 92-101.
18. Li J, He Y, Wang W, et al. Polyamine-Mediated Stoichiometric Assembly of Ribonucleoproteins for Enhanced mRNA Delivery. Angew Chem Int Ed Engl 2017; 56: 13709-13712.
19. Udhayakumar V K, De Beuckelaer A, McCaffrey J, et al. Arginine-Rich Peptide-Based mRNA Nanocomplexes Efficiently Instigate Cytotoxic T Cell Immunity Dependent on the Amphipathic Organization of the Peptide. Adv Healthc Mater 2017; 6. doi: 10.1002/adhm.201601412
20. Capasso Palmiero U, Kaczmarek J C, Fenton O S, et al. Poly(beta-amino ester)-co-poly(caprolactone) Terpolymers as Nonviral Vectors for mRNA Delivery In Vitro and In Vivo. Adv Healthc Mater 2018; 7: e1800249. doi: 10.1002/adhm.201800249.
21. Kowalski P S, Capasso Palmiero U, Huang Y, et al. Ionizable Amino-Polyesters Synthesized via Ring Opening Polymerization of Tertiary Amino-Alcohols for Tissue Selective mRNA Delivery. Adv Mater 2018: e1801151. doi: 10.1002/adma.201801151.
22. Kaczmarek J C, Kauffman K J, Fenton O S, et al. Optimization of a Degradable Polymer-Lipid Nanoparticle for Potent Systemic Delivery of mRNA to the Lung Endothelium and Immune Cells. Nano Lett 2018; 18: 6449-6454.
23. Qiu Y, Man R C H, Liao Q, et al. Effective mRNA pulmonary delivery by dry powder formulation of PEGylated synthetic KL4 peptide. J Control Release 2019; 314: 102-115.
24. Chan L Y, Khung Y L, Lin C Y Preparation of Messenger RNA Nanomicelles via Non-Cytotoxic PEG-Polyamine Nanocomplex for Intracerebroventicular Delivery: A Proof-of-Concept Study in Mouse Models. Nanomaterials (Basel) 2019; 9. doi: 10.3390/nano9010067
25. Patel A K, Kaczmarek J C, Bose S, et al. Inhaled Nanoformulated mRNA Polyplexes for Protein Production in Lung Epithelium. Adv Mater 2019; 31: e1805116. doi: 10.1002/adma.201805116.
26. Uchida S, Kinoh H, Ishii T, et al. Systemic delivery of messenger RNA for the treatment of pancreatic cancer using polyplex nanomicelles with a cholesterol moiety. Biomaterials 2016; 82: 221-228.
27. Leng Q, Mixson A J Modified branched peptides with a histidine-rich tail enhance in vitro gene transfection. Nucleic Acids Res 2005; 33: e40. doi: 10.1093/nar/gni040.
28. Blakney A K, Yilmaz G, McKay P F, et al. One Size Does Not Fit All: The Effect of Chain Length and Charge Density of Poly(ethylene imine) Based Copolymers on Delivery of pDNA, mRNA, and RepRNA Polyplexes. Biomacromolecules 2018; 19: 2870-2879.
29. Goncalves C, Akhter S, Pichon C, et al. Intracellular Availability of pDNA and mRNA after Transfection: A Comparative Study among Polyplexes, Lipoplexes, and Lipopolyplexes. Mol Pharm 2016; 13: 3153-3163.
30. Kauffman A C, Piotrowski-Daspit A S, Nakazawa K H, et al. Tunability of Biodegradable Poly(amine-co-ester) Polymers for Customized Nucleic Acid Delivery and Other Biomedical Applications. Biomacromolecules 2018; 19: 3861-3873.
31. Peng L, Wagner E Polymeric Carriers for Nucleic Acid Delivery: Current Designs and Future Directions. Biomacromolecules 2019; 20: 3613-3626.
32. Scholz C, Wagner E Therapeutic plasmid DNA versus siRNA delivery: common and different tasks for synthetic carriers. J Control Release 2012; 161: 554-565.
33. Bettinger T, Carlisle R C, Read M L, et al. Peptide-mediated RNA delivery: a novel approach for enhanced transfection of primary and post-mitotic cells. Nucleic Acids Res 2001; 29: 3882-3891.
34. Leng Q, Scaria P, Zhu J, et al. Highly branched HK peptides are effective carriers of siRNA. J Gene Med 2005; 7: 977-986.
35. Chou S T, Hom K, Zhang D, et al. Enhanced silencing and stabilization of siRNA polyplexes by histidine-mediated hydrogen bonds. Biomaterials 2014; 35: 846-855.
36. Midoux P, Monsigny M Efficient gene transfer by histidylated polylysine/pDNA complexes. Bioconjug Chem 1999; 10: 406-411.
37. Henig M, Geierstanger B Direct Detection of a Histidine-Histidine Side Chain Hydorogen Bond Important for Folding of Apomyoglobin. Journal of American Chemical Society 1999; 121: 5123-5126.
38. Putnam D, Gentry C A, Pack D W, et al. Polymer-based gene delivery with low cytotoxicity by a unique balance of side-chain termini. Proc Natl Acad Sci USA 2001; 98: 1200-1205.
39. Zhang L, Ambulos N, Mixson A J DNA delivery to cells in culture using peptides. Methods Mol Biol 2004; 245: 33-52.
40. Strober W Trypan Blue Exclusion Test of Cell Viability. Curr Protoc Immunol 2015; 111: A3 B 1-A3 B 3. doi: 10.1002/0471142735.ima03bs111.
41. Schindelin J, Arganda-Carreras I, Frise E, et al. Fiji: an open-source platform for biological-image analysis. Nat Methods 2012; 9: 676-682.
42. Chou S T, Leng Q, Scaria P, et al. Selective modification of HK peptides enhances siRNA silencing of tumor targets in vivo. Cancer Gene Ther 2011; 18: 707-716.
43. Leng Q, Woodle M C, Liu Y, et al. Silver adducts of four-branched histidine rich peptides exhibit synergistic antifungal activity. Biochem Biophys Res Commun 2016; 477: 957-962.
44. Chen Q R, Zhang L, Stass S A, et al. Co-polymer of histidine and lysine markedly enhances transfection efficiency of liposomes. Gene Ther 2000; 7: 1698-1705.
45. Leng Q, Chou S T, Scaria P V, et al. Buffering capacity and size of siRNA polyplexes influence cytokine levels. Mol Ther 2012; 20: 2282-2290.

1. A method for inducing cellular uptake of a mRNA molecule in vivo, comprising:
administering a HK polyplex, comprising a mRNA molecule bound to a HK polymer, to a mammal,
where the HK polymer is a polymer of Formula I or II 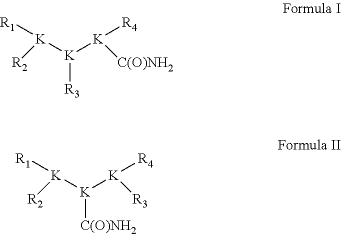
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

2. A method for inducing cellular uptake of a mRNA molecule in vivo, comprising:
administering a HK associated lipid particle, comprising a HK polymer, a lipid moiety, and a mRNA molecule, to a mammal,
where the HK polymer is a polymer of Formula I or II 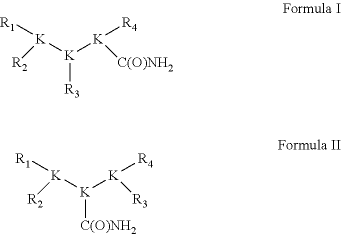
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

3. A method for inducing cellular uptake of a mRNA molecule in vitro or ex vivo, comprising:
culturing a HK polyplex, comprising a mRNA molecule bound to a HK polymer, with a target mammalian cell under conditions permitting uptake by the cell of the HK polyplex,
where the HK polymer is a polymer of Formula I or II 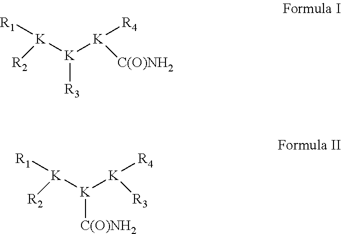
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

4. A method for inducing cellular uptake of a mRNA molecule in vitro or ex vivo, comprising:
culturing a HK associated lipid particle, comprising a HK polymer, a lipid moiety, and a mRNA molecule, with a target mammalian cell under conditions permitting uptake by the cell of the HK associated lipid particle,
where the HK polymer is a polymer of Formula I or II 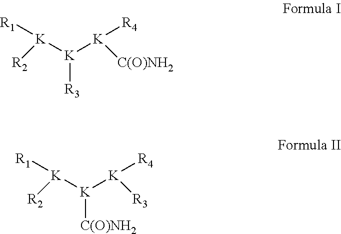
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

5. The method of claim 1, wherein the administration is local administration or systemic administration.

6. The method of claim 1, wherein each of R₁, R₂, R₃ and R₄ is the same and selected from R_{A}-R_{J}.

7. The method of claim 1, wherein each of R₁, R₂, R₃ and R₄ is the same and selected from R_{B}-R_{D}.

8. The method of claim 1, wherein the ratio of the mRNA molecule to the HK polymer is from 2:1 to 1:12 (wt:wt).

9. A method for preparing a HK associated lipid particle comprising:
(a) (i) mixing a mRNA molecule with a HK polymer under conditions permitting binding between the mRNA molecule and the HK polymer to form a HK polyplex, (ii) mixing the HK polyplex with a lipid moiety under conditions permitting binding between the HK polyplex and the lipid moiety to form a HK associated lipid particle; or
(b) (i) mixing a HK polymer with a lipid moiety under conditions permitting binding between the lipid moiety and the HK polymer, (ii) mixing the HK polymer-lipid of (i) with a mRNA molecule under conditions permitting binding between the mRNA molecule and the HK polymer-lipid to form a HK associated lipid particle; or
(c) (i) mixing a lipid moiety with a mRNA molecule under conditions permitting binding between the mRNA molecule and the lipid moiety, (ii) mixing the mRNA molecule-lipid complex of (i) with a HK polymer under conditions permitting binding between the mRNA molecule-lipid complex and the HK polymer to form a HK associated lipid particle.

10. The method of claim 9, wherein the HK polymer is associated with the lipid moiety by ionic, covalent, or hydrophobic interactions.

11. A method for preparing a HK associated lipid particle comprising mixing a mRNA molecule with a HK polymer conjugated with a lipid moiety (HK-lipid conjugate) under conditions permitting binding between the mRNA molecule and the HK-lipid conjugate to form a HK associated lipid particle.

12. The method of claim 9, wherein the HK associated lipid particle forms a micelle.

13. The method of claim 9, wherein the lipid moiety is one or more of a liposome, micelle, fatty acyl group, and cholesterol.

14. The method of claim 9, wherein the lipid moiety is a cationic lipid.

15. The method of claim 9, wherein the lipid moiety is DOTAP (1,2-dioleoyl-3-(trimethylammonium) propane), DOSPER (1,3-dioleoyloxy-2-(6-carboxy-spermyl)-propylamid), DOTMA (N-[1-(2,3-dioleyloxy)-propyl]-N,N,N-trimethylammonium chloride), DC-cholesterol, DLinDMA (an ionizable 1,2-dilinoleyloxy-N,N-dimethyl-3-aminopropane), or an imidazole and/or histamine liposome.

16-19. (canceled)

20. A method for inducing cellular uptake of a mRNA molecule, comprising targeting a mammalian cell with a HK associated lipid particle under conditions permitting uptake by the cell of the HK associated lipid particle, wherein the HK associated lipid particle comprises a mRNA molecule, a HK polymer of Formula I or II, and a lipid moiety 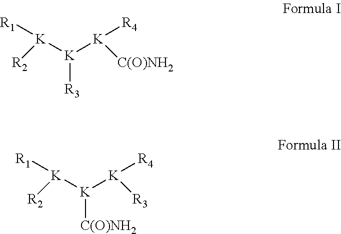
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine K is L-lysine and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-;
and wherein the lipid moiety is one or more of a liposome, micelle, fatty acyl group, and cholesterol.

21-33. (canceled)

34. A HK polyplex composition comprising a mRNA molecule bound by a HK polymer, where the HK polymer is a polymer of Formula I or II 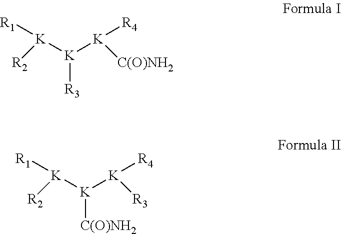
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

35. A HK associated lipid particle composition comprising a HK polymer, a lipid moiety, and a mRNA molecule, where the HK polymer is a polymer of Formula I or II 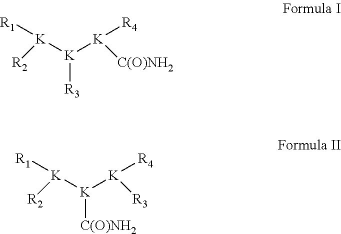
wherein in Formula I and II, K is the amino acid L-lysine and R₁, R₂, R₃ and R₄ are each individually selected from the group consisting of R_{A}-R_{J}, and wherein in R_{A}-R_{J}, H is L-histidine or D-histidine, K is L-lysine, and k is D-lysine
R_{A} =
(SEQ ID NO: 5)
KHKHHKHHKHHKHHKHHKHK-
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-
R_{H} =
(SEQ ID NO: 12)
KHHHKHHHKHHHKHHHHK-
R_{I} =
(SEQ ID NO: 13)
KHHHKHHHHKHHHKHHHK-
R_{J} =
(SEQ ID NO: 14)
KHHHKHHHHKHHHKHHHHK-.

36-46. (canceled)

47. A HK associated lipid particle composition comprising a HK polymer, a cationic lipid, and a mRNA molecule, where the HK polymer is a polymer of Formula I or II 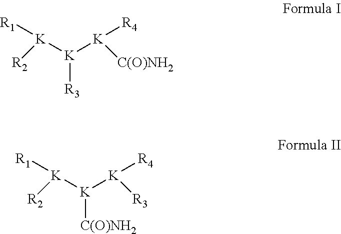
wherein in Formula I and II, K is the amino acid L-lysine and R₁, R₂, R₃ and R₄ are each the same and selected from the group consisting of R_{B}-R_{G}, and wherein in R_{B}-R_{G}, H is L-histidine or D-histidine, K is L-lysine, and k is D-lysine
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-
R_{E} =
(SEQ ID NO: 9)
HKHHHKHHHKHHHHKHHHK-
R_{F} =
(SEQ ID NO: 10)
HHKHHHKHHHKHHHHKHHHK-
R_{G} =
(SEQ ID NO: 11)
KHHHHKHHHHKHHHHKHHHHK-.

48. A HK associated lipid particle composition comprising a HK polymer, a cationic lipid, and a mRNA molecule, where the HK polymer is a polymer of Formula I or II 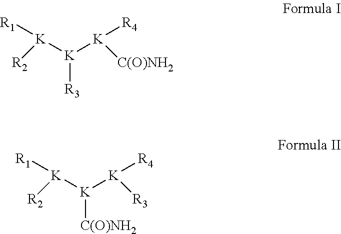
wherein in Formula I and II, K is the amino acid L-lysine and R₁, R₂, R₃ and R₄ are each the same and selected from the group consisting of R_{B}, R_{C}, and R_{D}, and wherein, in R_{B}, R_{C}, and R_{D}, H is L-histidine or D-histidine, K is L-lysine, and k is D-lysine
R_{B} =
(SEQ ID NO: 6)
KHHHKHHHKHHHKHHHK-
R_{C} =
(SEQ ID NO: 7)
KHHHKHHHKHHHHKHHHK-
R_{D} =
(SEQ ID NO: 8)
kHHHkHHHkHHHHKHHHk-.

49-56. (canceled)
